(19) United States
(12) Patent Application Publication
(10) Pub. No.: US 2009/0062741 A1
(43) Pub. Date: Mar. 5, 2009

(54) Dual lumen syringe

(76) Inventors: Emery Smith, Fort Myers, FL (US); Patrick Pennie, Fort Myers, FL (US); David K. Buzenius, Fort Myers, FL (US)

Correspondence Address: William E. Noonan, Post Office Box 07338, Fort Myers, FL, 33919, US

(21) Appl. No.: 11/985,204

(22) Filed: Nov. 14, 2007

Related U.S. Application Data
(60) Provisional application No. 60/858,818, filed on Nov. 14, 2006.

(51) Int. Cl. A61M 5/19

(52) U.S. Cl. 604/191

(57) ABSTRACT

A dual lumen syringe includes a body having a pair of elongate cavities or lumens formed therein. A directional valve is associated with each lumen. The valve is alternated between the first position wherein the lumen is communicably connected to a fluid inlet and a second position wherein the lumen is connected with a fluid outlet. There are a pair of elongate plungers, which are fixedly interconnected by a handle. Each plunger is received and longitudinally slidable in a reciprocating manner through a respective lumen. The valves are switched to a first position wherein the lumens are interconnected with the fluid inlets and the plunger is retracted to aspirate fluids through the respective inlets and into the lumens. The valves are then switched to a second position to communicably interconnect the lumens with the outlets. The plungers are then simultaneously driven inwardly through the respective lumens to drive the fluids simultaneously through the outlets to a dispensing tip permanently connected to the body and in communication with the outlets.

### RELATED APPLICATION

This application claims the benefit of U.S. Provisional Patent Application Ser. No. 60/858,818 filed Nov. 14, 2006.

### FIELD OF THE INVENTION

This invention relates to a dual lumen syringe capable of simultaneously aspirating a pair of fluids from respective containers and dispensing the fluids through a common outlet tip.

### BACKGROUND OF THE INVENTION

Medical syringes are typically fitted with a dispensing device such as a hypodermic needle or aerosol tip for first drawing or aspirating a fluid into the syringe and then dispensing that fluid by injecting it into or spraying it onto a patient. In certain applications, two different fluids must be utilized. This occurs, for example, in wound therapy wherein fibrinogen and thrombin are mixed and applied to a wound or other part of the body being treated. The process of applying such mixtures has been facilitated somewhat by the introduction of double-barrel syringes. These instruments allow the two fluids to be dispensed simultaneously from respective syringe barrels through a common applicator tip which effectively mixes and administers the fluids to the patient.

Traditional double-barrel syringes have utilized two separate and distinct syringe barrels mounted side-by-side on a frame or bracket. Before the barrels are mounted in the frame, a respective fluid is aspirated into the lumen or chamber of each syringe. A common handle interconnects the plungers of the respective syringes. By the same token, a common applicator tip is releasably attached to the Luer™ locks formed at the outlets or discharge ends of the respective syringe barrels. The applicator tip itself includes a Luer™ lock type outlet for engaging and carrying an aerosol spray tip or hypodermic needle. With the two syringes mounted in the frame, the handle is pushed to depress the plungers in unison. This causes the fluid to be dispensed simultaneously through the attached applicator tip in amounts proportional to the volumes of the respective syringe lumens.

Although the foregoing assembly does permit two fluids to be dispensed simultaneously, it exhibits a number of drawbacks. Every time the syringe is used, each fluid must be aspirated individually into its respective syringe. Then, the syringes must be tediously assembled in the frame. The common applicator tip must then be engaged with the syringes. Performing these individual steps can be awkward and take an undesirable amount of time, which tends to be a significant disadvantage in a medical or surgical setting. The user may have to search for appropriate syringes sizes required for a particular procedure. The fluids must then be aspirated and the syringes assembled in the frame. This requires an undesirable degree of fumbling, manipulation and maneuvering. The syringe barrels and other components can be dropped, lost, misplaced or easily contaminated.

Indeed, conventional double-barrel syringes often exhibit a significant risk of fluid contamination. Each of the syringe barrels aspirates and dispenses its respective fluid through the same port. Moreover, aspiration may be performed by a technician or person who is not sterile. When aspirated fluids are subsequently dispensed in a sterile environment, it is quite possible that the fluids may already be contaminated.

Manufacturing the individual components of known double-barrel syringes is also quite inefficient. The mounting frame, bracket or handle, the individual syringe barrels and the removable applicator tip are manufactured separately and independently of one another. A significant need exists for a simpler, more efficient and more cost effective manner of manufacturing a double-barrel syringe.

### SUMMARY OF THE INVENTION

It is therefore an object of the present invention to provide a dual lumen syringe that allows a pair of fluids to be quickly, efficiently and reliably aspirated and dispensed in desired proportions for a wide variety of medical, surgical and/or wound care applications.

It is a further object of this invention to provide a dual lumen syringe that significantly reduces the risk of fluid contamination.

It is a further object of this invention to provide a dual lumen syringe wherein separate syringe barrels are eliminated and instead a pair of separate lumens are formed in a single syringe body so that manufacture and assembly of the apparatus is improved considerably.

It is a further object of this invention to provide a dual lumen syringe that significantly reduces separable syringe parts and the related possibility that such parts may be misplaced or lost in a medical or surgical setting.

It is a further object of this invention to provide a dual lumen syringe that reliably and simultaneously draws and delivers precisely predetermined proportions of fluids.

It is a further object of this invention to provide a dual lumen syringe wherein the plungers, lumens and aspiration/applicator tip remain permanently assembled between uses such that it is much easier and faster to operate the syringe than prior double-barrel syringes.

It is a further object of this invention to provide a dual lumen syringe that may be reused repeatedly to aspirate and dispense a pair of fluids without having to change and/or disassemble individual syringes or syringe barrels between each use as has been required in the prior art.

It is a further object of this invention to provide a dual lumen syringe that is far easier, faster and more efficient to use than previous double-barrel syringes.

This invention results from a realization that a more efficient and easy to assemble and operate dual lumen syringe can be accomplished by employing a pair of elongate lumens or chambers formed integrally in the same syringe body. Such a syringe does not have to be fully disassembled and re-assembled each time it is used, as is the case with known devices of this type.

This invention features a dual lumen syringe including a syringe body having a pair of elongate lumens or chambers formed therein. Each lumen includes a relative diameter or volume that is proportional to the volume of fluid to be aspirated into and dispensed from the lumen. There are a pair of plungers that are engaged with the body such that each plunger is movable in a reciprocating manner within a respective lumen. A handle interconnects the respective plungers for enabling the plungers to be pushed and pulled simultaneously in a reciprocating manner through the respective lumens. Each plunger carries a seal that sealably interengages the wall of the lumen in which the plunger is moved. The seal allows a respective one of the plungers to slide longitudinally through the lumen in a selected longitudinal direction. Each lumen is communicably interconnected by an outlet channel to a common applicator or dispensing tip, which is operatively interengagable with a hypodermic needle, aerosol spray tip or other standard variety of fluid administering devices. Each lumen is also communicably connected to a separate inlet channel that in turn communicates with a selected supply of fluid. A first valve is operatively mounted to the syringe body for selectively connecting a first lumen to one of a first fluid supply and the outlet channel while blocking communication between the first lumen and the other of the first fluid supply and the outlet channel. Likewise, a second valve is operatively connected to the body for selectively interconnecting the second lumen to one of the second fluid supply and the outlet channel while blocking communication between the second lumen and the other of the second fluid supply and the outlet channel. Each valve is selectively switched into a first position to interconnect a respective lumen to an associated fluid supply. The plunger handle and interconnected plungers are then pulled outwardly from the lumens to simultaneously aspirate the fluids respectively through the valves and into the lumens. Each valve is then switched into a second position to block communication between the lumens and their respective fluid supplies and establish communication between the lumens and their respective outlet channels. The handle is depressed to drive the plungers inwardly through the lumens. This urges fluid from the lumens through the respective valves and outlet channels to the dispensing tip. The fluids are then dispensed simultaneously through the dispensing tip and attached fluid administering device.

In a preferred embodiment, each plunger includes at least one O-ring seal disposed peripherally about the plunger and between the plunger and an inside wall of the associated lumen. The dispensing tip may include a fluid transmitting applicator tip attached exteriorly to the body. A first outlet channel may extend through the body between a first one of the lumens and the dispensing tip. A second outlet channel may extend through the body between a second lumen and the dispensing tip. The dispensing tip may be permanently mounted to the body in communication with the outlet channels. The dispensing tip itself includes a channel formed therethrough for mixing and transmitting fluid from the outlet channels to the fluid administering device.

Each fluid supply may include a sealed container. A hose or tube may communicably interconnect each fluid supply with an inlet that is communicably connected to a respective lumen.

Typically, the valves feature directional valves. Each valve is mounted to the syringe body and alternated or switched between a pair of positions. In the first position, the valve opens the inlet and allows fluid from the connected fluid supply to be drawn into a respective lumen when the plunger is drawn outwardly from the lumen. At the same time, the valve blocks the outlet channel so that the fluid being aspirated from the fluid supply avoids the outlet channel.

When the valve is alternated to its second position, it allows its associated lumen to communicate with the outlet channel. At the same time, the fluid supply inlet channel is blocked by the valve. In this condition, the plunger may be pushed inwardly through the lumen so that fluid from that lumen is driven through the valve and communicating outlet channel to the hypodermic needle or other dispensing device.

Each directional valve may include a generally T-shaped valve opening. The valve may feature a cylindrical component having a valve handle extending generally radially therefrom. The valve handle may include an arrow or other indicia formed thereon. The handle and associated indicia are aligned with the outlet channel and point toward the dispensing tip to indicate that the valve is restricting the flow of fluid through the outlet channel to the dispensing tip. As a result, fluid may be aspirated from a fluid supply through the associated inlet channel and valve, and into the communicating lumen. Alternately, the valve handle is turned to rotate the valve into a second position wherein the arrow or other indicia is pointed toward the fluid supply inlet. This indicates that the outlet channel is open. The user presses the plunger to force fluid from the lumen and through the open valve for delivery through the outlet channel to the dispensing device.

In operation, the respective valves are similarly positioned, i.e. each valve is operated to open a respective inlet channel and close a respective outlet channel. The user then pulls the plungers outwardly from the body to simultaneously aspirate the two fluids into respective lumens. When aspiration is completed, the dual lumen syringe is utilized to administer the fluids by depressing the valve handle to push the plungers in unison inwardly through their respective lumens. This pushes the fluids in proportions determined by the relative sizes of the lumens through the open valves and the outlet channels to the dispensing tip and attached needle or sprayer, which administer the mixed fluids to a patient.

### DETAILED DESCRIPTION OF PREFERRED EMBODIMENTS

Other objects, features and advantages will occur from the following description of a preferred embodiment and the accompanying drawings, in which:

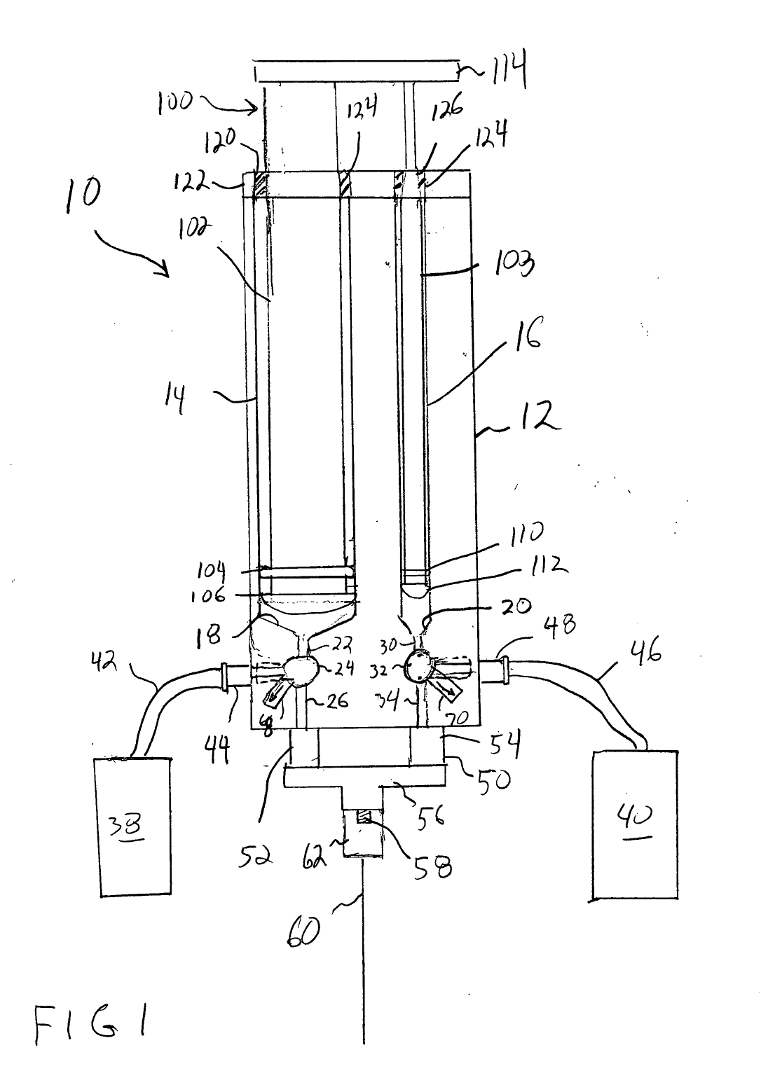

FIG. 1 is a front elevational view of a preferred dual lumen syringe in accordance with this invention, with the plunger assembly positioned for dispensing fluids from the syringe;

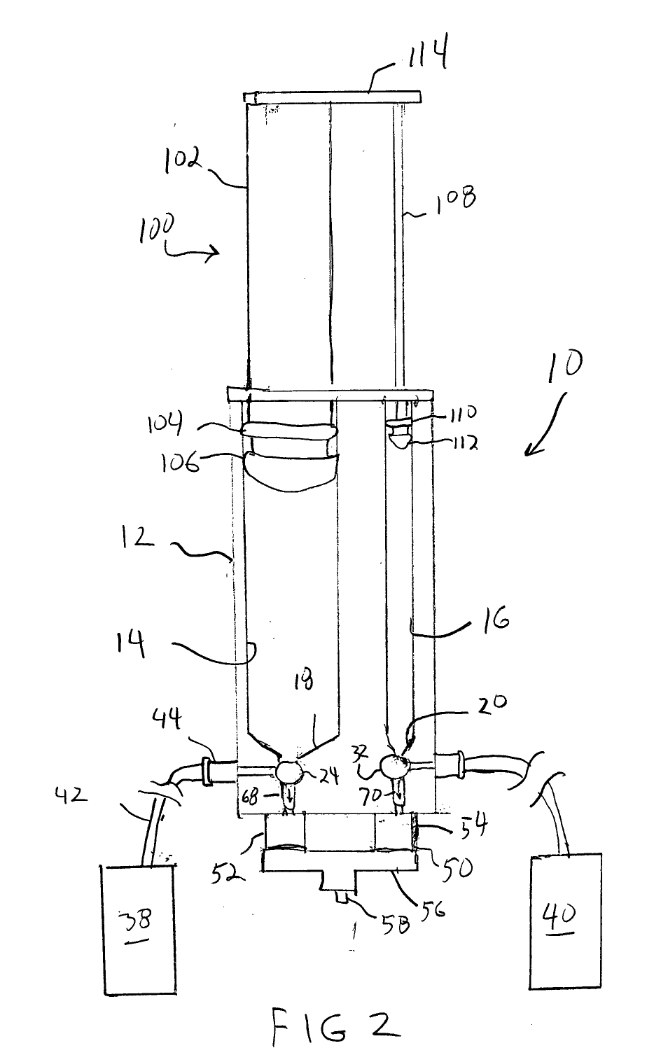

FIG. 2 is a front elevational view of the syringe with the plunger assembly positioned for aspirating fluids into the respective lumens;

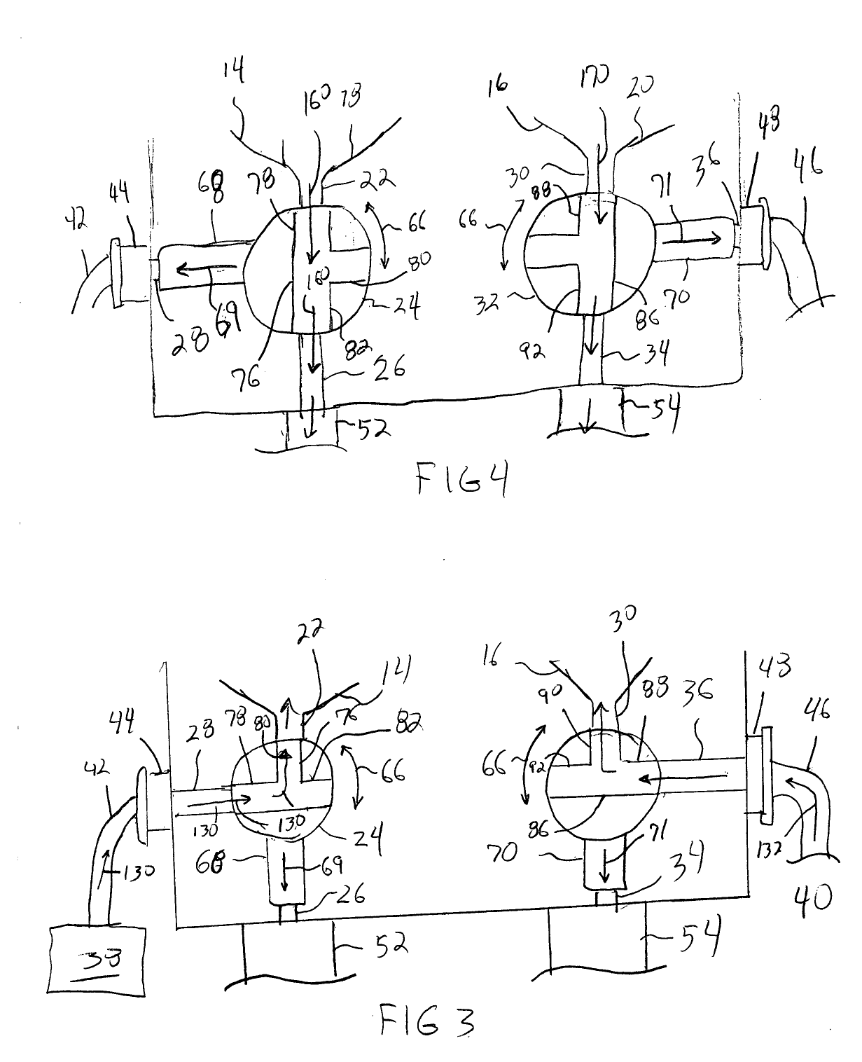

FIG. 3 is an elevational front view of the lower end of the syringe body with the directional valves depicted to clearly illustrate the fluid transmitting channel formed therein; the directional valves are specifically illustrated in respective first positions that open the lumens to their respective fluid supplies such that the fluids can be aspirated into the lumens; and

FIG. 4 is a view similar to FIG. 3 with the valves in their respective second positions wherein each lumen communicates with an associated outlet channel so that fluids may be dispensed by the syringe as the plungers are pushed inwardly through the lumens.

There is shown in FIGS. 1 and 2, dual lumen syringe 10 that is especially well suited for use in wound therapy, as well as other medical and surgical applications. Syringe 10 may be utilized to administer or apply any two selected types of fluid within the scope of this invention. For example, in wound therapy, the syringe may be used to inject or apply a mixture of fibrinogen and thrombin to a patient. Various other combinations of fluids may be mixed and dispensed using the syringe.

Syringe 10 includes an elongate body 12, which may be composed of polypropylene or various other types of plastics suited for use in medical equipment. Body 12 has a generally rectangular solid shape although other configurations may be employed within the scope of this invention.

An elongate pair of fluid accommodating cavities or lumens 14 and 16 are formed integrally within body 12. Each lumen extends from an upper end of the syringe body to a point relatively proximate the lower end of the body. In certain embodiments, body 12 may be transparent or translucent so that lumens 14 and 16 are visible, as shown herein. In alternative embodiments, the lumens may be obscured from view by the material composing body 12. Each lumen includes a generally cylindrical cross-sectional shape that terminates at a tapered lower discharge end 18, 20 respectively. This tapered lower end facilitates dispensing of the fluid from the lumen in the manner described more fully below. Lumens 14 and 16 may have various diameters for accommodating respective volumes of fluid therein. In the version depicted in FIGS. 1 and 2, lumen 14 has a volume that is 11 times greater than that of lumen 16. As a result, when fluids are mixed and dispensed by syringe 10, such fluids will be applied in a ratio of 11:1.

As further depicted in FIGS. 3 and 4, lower end 18 of lumen 14 communicates with a tubular passageway 22 within body 12. A first directional valve 24 selectively and communicably connects passageway 22 to either an outlet channel 26 or an inlet channel 28. The construction and operation of the valve are described more fully below. By the same token, lower end 20 of lumen 16 communicates with a tubular passageway 30 in the syringe body. That passageway is selectively interconnected by a second directional valve 32 to communicate with either a second outlet channel 34 or a second fluid inlet channel 36. Once again, the construction and operation of directional valve 32 is explained below.

Lumens 14 and 16, as well as tubular passageways 22, 30, inlet channels 28, 36 and outlet channels 26 and 34 are formed through syringe body 12 by various known machining techniques. The body may also be molded to include lumens and channels having selected dimensions and tolerances. By varying the sizes of the respective lumens, various selected proportions of fluids may be dispensed by the syringe. In alternative embodiments, the inlet and outlet channels, as well as the passageways may be replaced by alternative types of ports, orifices or other fluid transmitting conduits.

Referring to FIGS. 1-4, each lumen 14, 16 is communicably connected to a respective fluid supply 38 and 40. As previously indicated, the fluid supplies may comprise various types of fluids used in wound care, surgical and/or other medical or scientific applications. Each fluid supply 38, 40 is typically accommodated in a sealed container, which is connected in a sterile manner by a hose or tube to a respective inlet of syringe 10. For example, fluid 38 is communicably joined by a tube 42 to an inlet fitting 44 formed in the side of syringe body 12. Fitting 44 is communicably joined to inlet channel 28. Fluid 40 is likewise communicably connected to the syringe by a second tube 46. That tube is attached to a second inlet fitting 48 formed in the opposite side of syringe body 12. This inlet fitting in turn communicates with inlet channel 36.

As shown in FIGS. 1 and 2, a double syringe applicator or dispensing tip 50 is attached to the lower end of syringe body 12. Tip 50 closely resembles the removable type of double syringe applicator tip that is conventionally available. In particular, tip 50 includes a pair of discharge fittings 52, 54 and a body 56 that interconnects the fittings. The body itself carries a Luer™ lock discharge tip 58. Dispensing tip 50 largely employs a known construction. In particular, respective conduits or channels (not shown) are formed through fittings 52 and 54. These channels merge in an internal cavity (similarly not shown) in body 56, which effectively forms a mixing chamber. In the prior art, a conventional applicator of this general type is removably attached to the Luer™ lock discharge tips of respective syringe barrels such that each fitting, analogous to fittings 52 and 54, communicably engages a respective single barrel syringe tip. Fluids from the two syringes are then dispensed through the detachable fittings analogous to fittings 52 and 54 and into the mixing chamber of a body analogous to body 56. From there, the mixed fluids are dispensed through a Luer™ lock tip 58 (analogous to tip 50) to an attached applicator device such as aerosol sprayer or hypodermic syringe.

In the present invention, dispensing tip 50 is permanently attached to the lower end of syringe body 12 such that fittings 52 and 54 respectively communicate with outlet channels 26 and 34 formed through syringe body 12. Applicator 50 is attached to the body during manufacture of syringe 10. Unlike the prior art, the dispensing tip 50 is not removable, but instead, remains attached to the body both during and following use of the dual lumen syringe.

Tip 50 carries a fluid dispensing device in a manner comparable to the prior art. In particular, FIG. 1 depicts a hypodermic needle 60 that is attached to dispensing tip 50. A complementary Luer™ lock fitting 62 carried by needle 60 engages the Luer™ lock discharge tip 58. This secures the needle to the dispensing tip and establishes fluid communication between the tip 50 and needle 60. The outlet channels 26 and 34 formed through body 12 and permanently connected dispensing tip 50 effectively define a discharge outlet for the lumens 14 and 16. It should be understood that in alternative embodiments, various other types of fluid dispensing attachments, i.e. an aerosol spray tip, may be secured to dispensing tip 50 in an analogous manner.

As shown in FIGS. 1-4, directional valves 24 and 32 include generally cylindrical or circular configurations. Each valve is rotatably mounted within syringe body 12 such that the valve is axially rotatable as indicated by doubleheaded arrows 66 in FIGS. 3 and 4. More particularly, valve 24 includes a radial handle 68 that is attached circumferentially thereto. Handle 68 is turned to pivot or rotate valve 24 between the generally 6 o'clock position shown in FIG. 3 and the 9 o'clock position shown in FIG. 4. By the same token, valve 32 carries a handle 70 that extends radially outwardly from the circumference of valve 32. Handle 70 is operated to pivot valve 32 between the 6 o'clock position shown in FIG. 3 and the 3 o'clock position shown in FIG. 4. Handles 68 and 70 carry respective directional arrows 69 and 71, which function in a manner described more fully below.

Valves 24 and 32 typically comprise standard directional valves. Each valve includes a generally T-shaped channel that extends internally therethrough for permitting the valve to connect an associated lumen 14, 16 selectively to either inlet channel 36 or outlet channel 34. In this way, each lumen is able to communicate selectively with either the dispensing tip or its associated fluid supply.

Operation of valves 24 and 32 is best illustrated in FIGS. 3 and 4. Valve 24 includes T-shaped channel 76 comprising branches 78, 80 and 82. By the same token, valve 32 features a T-shaped channel 86 having branches 88, 90 and 92. When valve 24 is positioned in the manner shown in FIG. 3, with the handle 68 at a 6 o'clock position and indicator arrow 69 generally aligned with outlet channel 26, valve channel 76 is oriented such that channel branch 78 is aligned with inlet channel 28 and branch 80 is aligned with dispensing passageway 22 of lumen 14. Alternatively, when handle 68 is operated to pivot valve 24 into the second position shown in FIG. 4, with handle 68 in the 9 o'clock position and indicator arrow 69 aligned with inlet channel 28, valve channel 76 is oriented such that channel branch 78 is aligned with passageway 22 of lumen 14, and branch 82 is aligned with outlet channel 26. See FIG. 4.

Valve 32 operates in a similar manner. As shown in FIG. 3, handle 70 is positioned at 6 o'clock with indicator arrow 71 generally aligned with discharge channel 34. In this position, valve channel 86 is oriented so that branch 88 is aligned with inlet channel 36 and branch 90 is aligned with the passageway 30 formed at the bottom of lumen 16. Alternatively, as shown in FIG. 4, handle 70 may be operated to pivot valve 32 into a position wherein the handle is in a 3 o'clock position and indicator arrow 71 is aligned with inlet channel 36. In this position, branch 88 of valve channel 86 is aligned and in communication with lumen 16. Branch 92 is similarly aligned and communicates with discharge channel 34.

As best shown in FIGS. 1 and 2, a plunger assembly 100 is operatively interengaged with syringe body 12 and lumens 14 and 16 formed therein. In particular, plunger assembly 100 includes an elongate first plunger 102 that is slidably received within lumen 14. Plunger 102 carries a pair of O-rings 104 and 106 that peripherally surround the plunger and sealably interengage an interior wall of lumen 14. An elongate second plunger 108 is slidably receivable in lumen 16. Plunger 108 carries O-rings or other types of seals 110 and 112 that sealably interengage the interior wall of lumen 16. The seals define respective plunger diameters that correspond to the respective diameters of lumens 14 and 16.

Plungers 102 and 108 are fixedly interconnected by a bracket or handle 114. As best shown in FIG. 1, plunger 102 extends from handle 114 and through an opening 120 in top wall 122 of syringe body 12. An annular seal or bushing 124, shown in cross section, surrounds and seals plunger 102 and the surrounding opening in top wall 122. Likewise, plunger 108 extends through an opening 124 in top wall 122. A seal or bushing 126 interengages the plunger 108 and opening 124. As a result, plungers 102 and 108 are mounted for reciprocating longitudinal movement through respective lumens 14 and 16 between the extended or depressed plunger position shown in FIG. 1 and the retracted or aspirating plunger position shown in FIG. 2.

Dual lumen syringe 10 is operated in the following manner. The physician, medical technician, therapist or other user selects the fluids that are to be administered to a patient. Typically, such fluids must be administered or applied in predetermined proportions. The user selects a syringe having respective lumen diameters and volumes that will provide the required fluid proportion. Respective fluids 38 and 40 are then attached to the syringe by interconnecting tubes 42 and 46 to inlet fittings 44 and 48 respectively. Handle 114 is pressed or otherwise actuated to drive plungers 102 and 108 longitudinally inwardly through respective vacated lumens 14 and 16 until the plungers reach the positions generally shown in FIG. 1. If necessary, the user rotates directional valves 24 and 32 so that handles 68 and 70 point downwardly as shown in FIG. 2. As previously indicated, when indicator arrows 69 and 70 are positioned at 6 o'clock and the arrows are aligned with outlet channels 26 and 34, the valves 24 and 32 open the inlet channels 28, 36 and are positioned for the aspiration process. Specifically, as shown in FIG. 3, valve channel 76 is positioned to align inlet channel 28 with passageway 22 of lumen 14. By the same token, valve channel 86 communicably aligns inlet channel 36 with passageway 30 and lumen 16.

With the plunger assembly depressed and the valves 24 and 32 oriented in the foregoing manner, the user aspirates fluids 38, 40 by retracting handle 114. The plungers 102 and 108 are drawn outwardly from respective lumens 14 and 16 in the manner shown in FIG. 2. A vacuum is drawn within each lumen, which draws fluids 38 and 40 into the syringe. Specifically, as shown by arrow 130 in FIG. 3, fluid 38 is drawn through tube 42, inlet fitting 44, inlet channel 28, valve channel 76, and passageway 22 into lumen 14. Similarly, as indicated by arrows 132, fluid 40 is drawn through tube 46, inlet fitting 48, inlet channel 36, valve channel 86, and passageway 30 into lumen 16. The plunger assembly is retracted until the selected volumes of fluid are drawn into the respective lumens 14 and 16. Aspiration is then complete. During the aspiration process, valves 24 and 32 close the outlet channels and prevent aspirated fluid from entering the dispensing tip.

Upon completion of fluid aspiration, directional valves 24 and 32 are then switched. Handle 68 of valve 24 is rotated from the 6 o'clock position shown in FIG. 3 to the 9 o'clock position shown in FIG. 4. By the same token, handle 70 of valve 34 is rotated from the position shown in FIG. 3 to the position shown in FIG. 4. With the valves in the position shown in FIG. 4, the previously aspirated fluids are blocked from flowing out through the inlet channels 28 and 30 and thereby back into the fluid supplies. Rather, valve 24 and 32 are now oriented such that lumen 14 communicates through valve channel 76 with outlet channel 26. By the same token, lumen 16 communicates through valve channel 86 with outlet channel 34.

With the valves repositioned as shown in FIG. 4, the user is able to administer the fluids to the patient. Specifically, the user depresses handle 14 so that plungers 102 and 108 are pushed or advanced inwardly through respective lumens 14 and 16. The previously aspirated fluids are then driven through aligned valve channels 76 and 86 as shown by arrows 160 and 170 in FIG. 4. Specifically, fluids 38 and 40 pass through aligned valve channels 76 and 86 respectively and into outlet channels 26 and 34. From there, the fluid is transmitted through fittings 52 and 54 of dispensing tip 50. The fluids combine in the mixing chamber of section 56 and are discharged through Luer™ lock tip 58 and the attached dispensing device (i.e. hypodermic needle 60). The combined fluids are thereby applied or administered to a patient to perform the required therapy.

Syringe 10 allows combined fluids to be administered quickly, efficiently and in an extremely sterile manner. Because the two lumens are formed integrally in the body of the syringe, the syringe does not have to be disassembled and re-assembled and separate syringe barrels do not have to be replaced in a holder each and every time the syringe is used. Instead, the dual lumen syringe can be re-used without having to change syringe barrels. As a result, the administration of fluids is expedited, facilitated and made much more efficient.

Sterility is improved because the syringe allows the fluids to be aspirated directly from sealed containers. There is much less risk of contamination than is presented by using known double-barrel syringes. Sterility is further enhanced because, unlike known syringes, the present device eliminates aspirating and injecting fluids through the same port of the syringe.

The construction and assembly of syringe 10 also represents a significant improvement over known syringes. The dispensing tip is attached permanently rather than removably to the syringe body. Moreover, the lumens are formed integrally within the same body. Two separate syringe barrels and a holder do not have to be built. Not only does this simplify, facilitate and reduce the cost of constructing the syringe, it also significantly reduces the delays and annoyance that commonly occur when individual pieces of a conventional double-barrel syringe (i.e. individual syringe barrels, holders, applicator tips) are misplaced or lost. The user no longer has to fumble to tediously assemble and disassemble the syringe each time it is used. A simpler, easier to use and much more cost efficient syringe is provided.

From the foregoing it may be seen that the apparatus of this invention provides for an improved dual lumen syringe. While this detailed description has set forth particularly preferred embodiments of the apparatus of this invention, numerous modifications and variations of the structure of this invention, all within the scope of the invention, will readily occur to those skilled in the art. Accordingly, it is understood that this description is illustrative only of the principles of the invention and is not limitative thereof.

Although specific features of the invention are shown in some of the drawings and not others, this is for convenience only, as each feature may be combined with any and all of the other features in accordance with this invention.

Other embodiments will occur to those skilled in the art and are within the following claims:

What is claimed is:

1. A dual lumen syringe comprising:
a syringe body having elongate first and second lumens formed therein, each lumen having a volume proportional to a volume of fluid to be aspirated into and dispensed from said lumen;
a pair of plungers engaged with said body, each plunger capable of reciprocating longitudinal movement within a respective lumen;
a handle interconnecting said plungers and for enabling said plungers to be pushed and pulled in reciprocating longitudinal movement through said respective lumens;
each said lumen communicably connected through a respective inlet to a respective fluid supply and through a common dispensing outlet to a fluid administering device;
a first valve operatively mounted to said syringe body for selectively establishing communication between said first lumen and one of a first fluid supply and said common dispensing outlet while blocking communication between said first lumen and the other of said first fluid supply and said common dispensing outlet; and
a second valve operatively connected to said syringe body for selectively establishing communication between said second lumen and one of a second fluid supply and said common dispensing outlet while blocking communication between said second lumen and the other of said second fluid supply and said common dispensing outlet;
said valves being selectively disposed in a first position to establish communication between said first and second lumens and said first and second fluid supplies, respectively, and said handle being operated to pull said plungers outwardly through said lumens to aspirate fluids from said first and second fluid supplies simultaneously through said valves and into said respective first and second lumens;
said valves being alternately disposed in a second position to establish communication between said lumens and said common dispensing outlet and said handle being operated to drive said plungers inwardly through said lumens whereby fluids from said lumens are simultaneously delivered through said common dispensing outlet to said fluid administering device.

2. The apparatus of claim 1 in which each said plunger carries at least one o-ring seal for slidably and sealably interengaging an inside wall of said lumen in which said plunger is disposed.

3. The apparatus of claim 1 in which said common dispensing outlet includes a mixing chamber communicably connected to each said lumen for mixing fluids from said first and second lumens therein.

4. The apparatus of claim 3 further including a first outlet channel extending through said body between said first lumen and said mixing chamber and a second outlet channel between said second lumen and said mixing chamber.

5. The apparatus of claim 1 in which said discharge outlet is formed in a fluid transmitting dispensing tip attached to said body.

6. The apparatus of claim 5 in which said fluid transmitting dispensing tip includes a fluid transmitting conduit formed therethrough.

7. The apparatus of claim 6 in which said fluid transmitting conduit includes a mixing chamber for mixing fluids from said first and second fluid supplies therein and for transmitting fluid from said outlet channels to said discharge outlet.

8. The apparatus of claim 1 in which each fluid supply includes a sealed container joined to a respective lumen inlet by a respective inlet tube.

9. The apparatus of claim 1 in which said valves include directional valves, each alternatable between said first and second positions to enable a respective said plunger to effectively aspirate fluid into and dispense fluid from a respective one of said first and second fluid supplies in response to said handle being operated to pull said plungers outwardly and push said plungers inwardly through said lumens respectively.

10. The apparatus of claim 1 in which each valve has a T-shaped passageway.

11. The apparatus of claim 1 in which each said valve includes a rotatable hub having a valve passageway formed therethrough.

12. The apparatus of claim 11 in which each said hub carries a valve handle extending radially from said hub.

13. The apparatus of claim 1 in which each valve includes indicia to reflect the selected positions in which said valve is disposed.

14. The apparatus of claim 1 in which each valve carries a directional arrow for being directed generally toward said inlet of an associated said lumen to indicate that said valve is establishing communication between that lumen and an associated fluid supply and for being directed generally at said outlet channel of said associated lumen to indicate that said valve is establishing fluid communication between said associated lumen and said dispensing outlet.

14. The apparatus of claim 11 in which each valve carries a directional arrow for being directed generally toward said inlet of an associated said lumen to indicate that said valve is establishing communication between that lumen and an associated fluid supply and for being directed generally at said outlet channel of said associated lumen to indicate that said valve is establishing fluid communication between said associated lumen and said dispensing outlet; said directional arrow being disposed on said hub, said valve including a T-shaped fluid passageway formed through said hub.

15. A dual lumen syringe comprising:
a syringe body having elongate first and second lumens formed therein, each lumen having a volume proportional to a volume of fluid to be aspirated into and dispensed from said lumen;
a pair of plungers engaged with said body, each plunger capable of reciprocating longitudinal movement within a respective lumen;
a handle interconnecting said plungers and for enabling said plungers to be pushed and pulled in reciprocating longitudinal movement through said respective lumens;
each said lumen communicably connected through a respective inlet to a respective fluid supply and through a common dispensing outlet to a fluid administering device;
a first valve operatively mounted to said syringe body for selectively establishing communication between said first lumen and one of a first fluid supply and said common dispensing outlet while blocking communication between said first lumen and the other of said first fluid supply and said common dispensing outlet; and
a second valve operatively connected to said syringe body for selectively establishing communication between said second lumen and one of said second fluid supply and said common dispensing outlet while blocking communication between said second lumen and the other of said second fluid supply and said common dispensing outlet.
